(19) United States
(12) Plant Patent Application Publication
(10) Pub. No.: US 2008/0320630 P1
(43) Pub. Date: Dec. 25, 2008

(54) Impatiens plant named 'Fify Lave07'

(76) Inventor: Erik Smit, Andijk (NL)

Correspondence Address: JONDLE & ASSOCIATES P.C., 858 HAPPY CANYON ROAD SUITE 230, CASTLE ROCK, CO, 80108, US

(21) Appl. No.: 11/821,679

(22) Filed: Jun. 25, 2007

(51) Int. Cl. A01H 5/00

(52) U.S. Cl. PLT/318.6

(57) ABSTRACT

A new Impatiens plant particularly distinguished by single, violet flowers, compact, upright, outwardly spreading and mounded plant habit, free flowering, good basal branching, and moderately vigorous growth habit, is described.

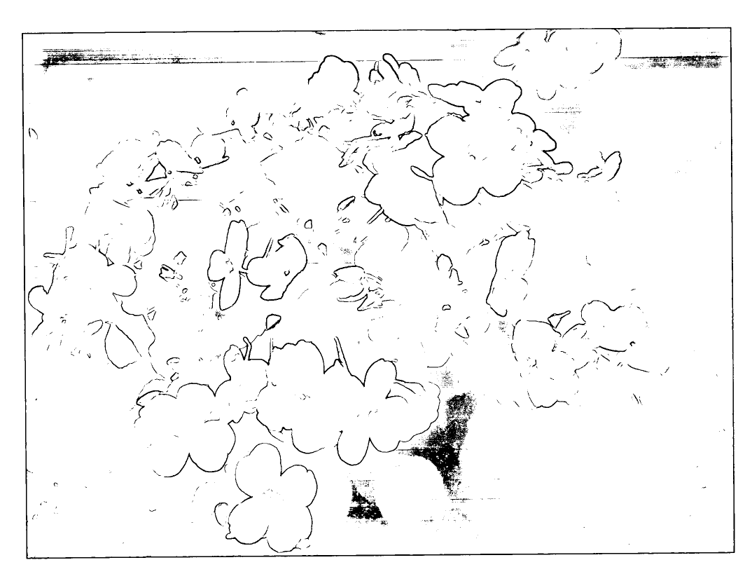

### GENUS AND SPECIES

Impatiens walleriana

### VARIETY DENOMINATION

‘Fify Lave07’

### BACKGROUND OF THE NEW PLANT

The present invention comprises a new and distinct cultivar of Impatiens, botanically known as Impatiens walleriana, and hereinafter referred to by the cultivar name ‘Fify Lave07’. The new cultivar originated from a hybridization made in August 2003 in Andijk, The Netherlands. The female parent was the proprietary violet flowered Impatiens plant ‘IMW-776-4’ (unpatented), while the male parent was the proprietary violet with white eye flowered Impatiens plant ‘IMY-31-3’ (unpatented). The seeds produced by the hybridization were sown in November 2003. A single plant selection was chosen for further evaluation and for asexual propagation in February 2004.

The new cultivar was created in 2003 and has been asexually reproduced repeatedly by vegetative cuttings and tissue culture in Andijk, The Netherlands, Hillschied, Germany and Gilroy, Calif. over a two year period. The plant has also been trialed at Gilroy, Calif., Andijk, The Netherlands and Hillscheid, Germany. The present invention has been found to retain its distinctive characteristics through successive asexual propagations.

Plant Breeder's Rights for this cultivar have been applied for in Europe on Nov. 13, 2006.

### SUMMARY OF THE INVENTION

The following are the most outstanding and distinguishing characteristics of this new cultivar when grown under normal horticultural practices in Gilroy, Calif. and Andijk, The Netherlands.
1. Single, violet flowers;
2. Compact, upright, outwardly spreading and mounded plant habit; and
3. Free flowering, good basal branching and moderately vigorous growth habit.

### DESCRIPTION OF PHOTOGRAPH

This new Impatiens plant is illustrated by the accompanying photograph which shows the overall plant habit including blooms, buds, and foliage of the plant; the colors shown are as true as can be reasonably obtained by conventional photographic procedures. The photograph is of a whole plant about 9 to 10 weeks old, grown in a greenhouse in Hillscheid, Germany during the spring. The photograph was taken in May 2006 and shows the overall plant habit including blooms, buds, and foliage.

### DESCRIPTION OF THE NEW CULTIVAR

The following detailed descriptions set forth the distinctive characteristics of ‘Fify Lave07’. The data which defines these characteristics were collected from asexual reproductions carried out in Gilroy, Calif. in a spring trial setting. The plant history was taken on 20-week-old plants grown in 8-inch fiber display pots in a greenhouse in the early spring. The plants had one plant growth regulator and one terminal pinch after cuttings were transplanted. Color readings were taken under natural light in the greenhouse. Color references are primarily to the R.H.S. Colour Chart of The Royal Horticultural Society of London (R.H.S.) (2001). Texture description data was viewed with a dissecting microscope.

### DETAILED BOTANICAL DESCRIPTION OF THE NEW PLANT

Classification:
   Family.—Balsaminaceae.
   Botanical.—Impatiens walleriana.
   Common name.—Impatiens.
Parentage:
   Female parent.—‘IMW-776-4’ a proprietary violet flowered Impatiens plant (unpatented).
   Male parent.—‘IMY-31-3’ a proprietary violet with white eye flowered Impatiens plant (unpatented).
Growth:
   Form.—Compact, upright, outwardly spreading and mounded plant habit.
   Growth and branching habit.—Free flowering, good basal branching, moderately vigorous, pinching of the terminal apices enhances branching.
   Height (from soil level to top of blooms).—10.0 cm to 12.0 cm.
   Width (including flowers).—20.0 cm to 22.0 cm.
   Time to produce a finished flowering plant.—About 8 to 10 weeks for a 4-inch pot.
   Outdoor plant performance.—Use in shade to semi-shade, use in hanging baskets, mixed containers, window boxes and mass plantings.
   Time to initiate and develop roots.—Approximately 10 to 21 days.
   Root description.—Fibrous, fleshy, white.
Branches:
   Stem.—Color: Closest to RHS 144C Length: 14.0 cm to 18.0 cm Diameter: 0.5 cm Internode length: 1.0 cm to 1.5 cm Texture: Smooth, glabrous Anthocyanin: Only in the older nodes of stems; closest to but a muted hue of RHS 175A.
   Peduncle.—Color: Closest to RHS 144C Length: 1.3 cm to 1.4 cm Diameter: 0.15 cm Texture: Smooth, glabrous.
   Pedicel.—Length: 1.6 cm to 1.8 cm Diameter: 0.1 cm Color: Closest to RHS 144C Texture: Smooth, glabrous.
Leaves:
   Arrangement.—Alternate, whorls.
   Size.—Length: 2.5 cm to 2.8 cm Width: 1.5 cm to 2.1 cm.
   Shape.—Elliptical to broadly elliptical (almost ovate).
   Apex.—Acute.
   Base.—Attenuate.
   Margin.—Crenate, with ciliation.
   Color (both immature and mature).—Upper surface: Closest to RHS 137A Lower surface: Closest to RHS 138B.
   Texture.—Smooth, glabrous.
   Venation color.—RHS 144C basally, and becoming indistinguishable within the leaf itself.
   Petiole.—Length: 0.7 cm to 2.5 cm Diameter: 0.2 cm to 0.3 cm Texture: Smooth, glabrous Color: RHS 144C.
Flower buds:
   Shape.—Ovate.
   Size.—Length: 0.7 cm to 0.8 cm Diameter: 0.5 cm to 0.6 cm.
   Color at tight bud.—RHS 69C.
Inflorescence:
   Blooming habit.—Continuous and freely all through the year.
   Type.—Racemes in leaf axis and in leaf whorls.
   Number of flowers per lateral branch.—30, including buds.
   Lastingness of the individual blooms on the plant.—About 5 to 7 days in the greenhouse.
   Fragrance.—None.
Flowers:
   Form.—Single.
   Corolla size.—Width: 2.6 cm to 2.8 cm Depth: 2.7 cm to 2.8 cm.
Petals:
   Number of petals per flower.—5; 1 standard, dorsal (free); 2 lateral or lobed pairs (wing petals) deeply lobed.
   Dorsal.—Length: 1.0 cm Width: 1.2 cm to 1.4 cm Shape: Broadly obovate Apex: Slightly emarginate Color: Immature: Upper surface: Lightly mottled, and lighter than RHS 75A Lower surface: RHS 76C, with a keel of RHS 143C Mature: Upper surface: Closest to RHS 76B but with a little more pink Lower surface: RHS 76C with a keel of RHS 143C.
   Lateral.—Length: 1.2 cm to 1.4 cm Width: 0.9 cm to 1.0 cm Shape: Obovate Apex: Slightly emarginate Color: Immature: Upper surface: RHS 75A with a slight spot of RHS N78A and RHS 155C basally Lower surface: RHS 76C Mature: Upper surface: RHS 75A with small spots of RHS N78A and RHS 155C basally Lower surface: RHS 76C.
   Base.—Attenuate to fused.
   Margin.—Entire.
   Texture.—Smooth, glabrous.
Sepals:
   Quantity.—3; 1 larger than the others with a spur.
   Texture.—Smooth, glabrous.
   Large sepals.—Color: Closest to RHS 155C Length: 0.6 cm to 0.8 cm Width: 0.5 cm to 0.6 cm Shape: Ovate Apex: Acute Base: Fused Margin: Entire Spur: Color: Lighter than RHS 1D Length: 2.5 cm to 2.7 cm Diameter: 0.2 cm.
   Small sepals.—Color: RHS 43B Length: 0.2 cm Width: 0.1 cm Shape: Lanceolate Apex: Acute Margin: Entire Base: Fused Texture: Smooth, glabrous.
Reproductive organs:
   Stamen.—5; more or less fused by the anthers, and covering the ovary.
   Anther length.—0.2 cm.
   Anther color.—RHS 58A.
   Pollen amount.—Moderate.
   Pistil.—1.
   Pistil length.—0.3 cm.
   Style color.—RHS 155A.
   Stigma color.—RHS 147A.
Fruit and seed set: Seed set not observed
Disease and insect resistance: No particular resistance or susceptibility has been observed.

### COMPARISON WITH PARENTAL AND COMMERCIAL CULTIVARS

‘Fify Lave07’ differs from the female parent ‘IMW-776-4’ in that ‘Fify Lave07’ has a smaller flower size and a more compact plant habit than ‘IMW-776-4’.

‘Fify Lave07’ differs from the male parent ‘IMY-31-3’ in that ‘Fify Lave07’ has a smaller flower size and a different flower color than ‘IMY-31-3’. Additionally, ‘Fify Lave07’ has a more compact plant habit and is more free-flowering than ‘IMY-31-3’.

‘Fify Lave07’ differs from the commercial cultivar ‘Balpixropi’ (U.S. Plant Pat. No. 15,213) in that ‘Fify Lave07’ has a lighter bud color with a lighter violet flower color than ‘Balpixropi’. Additionally, ‘Fify Lave07’ has darker green pedicles and lighter green foliage with a longer spur than ‘Balpixropi’.

I claim:

1. A new and distinct cultivar of Impatiens plant as shown and described herein.
